(19) United States
(12) Patent Application Publication
(10) Pub. No.: US 2010/0047283 A1
(43) Pub. Date: Feb. 25, 2010

(54) FRANCISELLA STRAIN FOR LIVE VACCINE

(76) Inventors: Stephen Lloyd Michell, Wiltshire (GB); Petra Claire Farquar Oyston, Wiltshire (GB); Mark Ivor Richards, Wiltshire (GB); Richard William Titball, Wiltshire (GB)

Correspondence Address: JOHN S. PRATT, ESQ;KILPATRICK STOCKTON, LLP, 1100 PEACHTREE STREET, SUITE 2800, ATLANTA, GA, 30309, US

(21) Appl. No.: 12/520,545

(22) PCT Filed: Dec. 21, 2007
(86) PCT No.: PCT/GB2007/004930

§ 371 Date: Jul. 14, 2009

(30) Foreign Application Priority Data
Dec. 21, 2006 (GB) 06255871

(51) Int. Cl. A61K 39/02; C12N 1/20; A61P 31/04

(52) U.S. Cl. 424/234.1; 435/252.1

(57) ABSTRACT

A strain of Francisella species in which gene FTT1564, or homologue thereof, has been inactivated, and which is able to produce a protective immune response in an animal, for use as live prophylactic or therapeutic vaccine against infection by said Francisella species. Other embodiments of the invention describe strains which comprise a further genetic mutation wherein additional genes are also inactivated. Pharmaceutical compositions comprising said strains, together with methods of preventing and treating tularemia infection, which utilise such strains, are also described and claimed.

This invention relates to live strains of Francisella species, their use as prophylactic or therapeutic vaccines, to compositions comprising these strains, and their use in the prevention or treatment of bacterial infection.

Francisella tularensis is an extremely pathogenic Gram-negative bacterium and is the etiological agent of the zoonotic disease Tularemia. There are four recognised sub-species of F. tularensis, including subspecies tularensis, holarctica, and novicida, which exhibit a high degree of genetic conservation. The most virulent subspecies is Francisella tularensis subspecies tularensis, which has an infectious dose in humans of as little as 10 cells via the airbourne route.

At present there is no available vaccine against F. tularensis infection although it has been demonstrated previously that an undefined attenuated strain of Francisella tularensis, which has been designated Francisella tularensis live vaccine strain (LVS), is capable of providing protection against the most virulent subspecies, tularensis. However, the LVS vaccine is not registered and has only been used to vaccinate at-risk individuals under special license. This license has now been withdrawn. The LVS strain is likely to remain unlicensed because the genetic changes that are responsible for the attenuating phenotype are not understood at the molecular level. Therefore, there exists a possibility that the vaccine strain could revert back to the fully virulent form. Further, it has been shown that whilst LVS provides effective protection in the mouse model of infection, protection is not complete. The protection afforded by LVS against an aerosol challenge of the most virulent tularensis subspecies is sub-optimal. Clearly, a vaccine which is genetically stable and which provides complete protection is highly desirable.

The fact that naturally occurring, attenuated strains of F. tularensis can induce protective immunity does suggest that an attenuated strain with properly defined genetic mutations in the organism's virulence factors is a feasible approach in vaccine development.

Unfortunately, however, relatively little is known about the virulence mechanisms of F. tularensis and, as such, virulence factors have proven to be very difficult to predict; the recent completion of the genome sequence of the virulent strain F. tularensis subspecies tularensis SchuS4 has so far failed to reveal the presence of classical virulence factors such as toxins or type-III secretion systems, which are predominant in many other pathogenic bacteria.

Some efforts to identify new vaccine strains have focused on naturally occurring strains (other than LVS) or on spontaneously attenuated strains of F. tularensis, such as the FSC043 mutant of SchuS4 reported by Twine et al (Infection and Immunity Vol 73, 2005, pp 8345-8352). Examination of these attenuated mutants has confirmed that mutations can lead to attenuated strains which afford some level of protection against tularemia. However, the molecular basis of this attenuation and protection is unknown and, in any case, the protection afforded is not better than that provided by LVS. The problem remains, therefore to find defined mutations which give complete protection against the most virulent forms of Francisella.

A live vaccine strain of Francisella derived from the subspecies novicida, which contains a single genetic mutation, is described in co-pending International Application number PCT/GB2004/001264. An alternative live vaccine strain of Francisella tularensis subspecies tularensis strain SchuS4 is described in co-pending British patent application number GB0614743.3. These applications show that a single genetic lesion in either the purine enzyme pathway or the synthetic pathway for the production of the capsule component of the bacterial cell provide attenuated strains which are also protective in the mouse model of infection. Whilst this clearly represents significant advances towards the creation of a vaccine for tularemia, it is widely recognised that at least one additional genetic mutation would be required to enable either of these strains to be developed and to obtain licensed status. The problem remains, therefore, to determine alternative and/or additional mutations which provide live strains of Francisella tularensis which are attenuated but which also provide complete protection against all strains of Francisella tularensis.

New live vaccines, containing well defined mutations and which are fully protective against tularemia are therefore required.

The applicants have found that by modifying strains of Francisella in a particular way, attenuated strains which are protective can be produced. These live strains can be used as the basis for new vaccines against tularemia.

The present invention therefore provides a strain of Francisella species wherein a gene which encodes for a hypothetical protein, chosen from the genome of the highly virulent SchuS4 strain, has been inactivated, and which is able to produce a protective immune response in an animal, for use as a live prophylactic or therapeutic vaccine against infection by Francisella species.

In particular, the strain has gene FTT1564, or a homologue of this gene, inactivated.

As used herein, the terms “homologue”, “homologous” and “homology” mean, at the protein level, the similarity of two amino acid sequences are such that the two sequences share greater than 30% identity. Identity in this instance can be judged for example using the BLAST program (vs. 2.2.12) found at http://www.ncbi.nlm.nih.gov/BLAST/ or the algorithm of Lipman-Pearson with, for example, Ktuple: 2, gap penalty: 4, Gap Length Penalty: 12, standard PAM scoring matrix or other suitable parameters as readily determined by a person skilled in the art (Lipman, D. J. and Pearson, W. R., Rapid and Sensitive Protein Similarity Searches, Science, 1985, vol. 227, 1435-1441).

Gene FTT1564 may be readily determined from the genome sequence of Francisella tularensis and suitable homologues in subspecies may be readily determined by analysis of the relevant genome sequence using either or the above methods. In particular, a corresponding gene in Francisella tularensis subspecies novicida is that denoted by FTN1472.

Inactivation of the gene can be carried out using any of the conventional methods known in the art. Typically, the strain is transformed with a vector which has the effect of down-regulating or otherwise inactivating the gene. This can be done by mutating control elements such as promoters and the like which control gene expression, by mutating the coding region of gene so that any product expressed is inactive, or by deleting the gene entirely. Alternatively, the gene can be inactivated at the RNA or protein level, by transforming the cell so that it expresses a sense or anti-sense construct which binds to DNA or RNA encoding the gene to prevent transcription thereof.

Preferably however, the gene is inactivated by complete or partial deletion mutation or by insertional mutation.

The applicants have found that it is preferable to inactivate the FTT11564 gene or its homologue (such as FTN1472) from Francisella species such as F. tularensis subspecies novicida or subspecies tularensis. In particular, it is preferred that FTT1564 is inactivated in F. tularensis subspecies tularensis strain SchuS4 to provide a preferred strain of the present invention.

In particular, the applicants have found that strains of Francisella species which have the FTT1564 gene, or a homologue this gene inactivated are attenuated. As illustrated hereinafter, a strain of F. tularensis subspecies tularensis which has an inactivated FTT1564 gene is protective, in mice, against an aerosol challenge with Francisella novicida.

The strain of the invention suitably has a further defined mutation or lesion so as to reduce the risk of the bacterium reverting to a virulent form. In this case, the mutation is in a gene which is selected so that the strain is suitably attenuated, but can still retain the ability to stimulate a sufficient immune response to provide long term protection. Suitable additional mutations can be identified using conventional methods, and examination and analysis of the current live vaccine strain (LVS) or other attenuated strains may assist in the identification. Examples of these mutations include, but are not limited to, mutations to the FTT0808, FTT0918 or FTT0919 genes. Alternatively, the further defined mutation may advantageously involve a gene which encodes for cell surface components or enzyme pathways utilised within the bacterial cell. Such further mutations include, but are not limited to, mutations which inactivate genes which encode the capsule component of the cell, such as capB and/or capC or pilin genes, for example mutations to pilA and/or pilE and/or pilC genes or other mutations such as those described in co-pending British patent application number GB0511722.1 (the contents of which are hereby incorporated by reference), mutations which inactivate genes which encode enzymes in the purine pathway, for example mutations to purA and/or purF genes and other purine pathway genes such as those described in co-pending International Application number PCT/GB2004/001264 (the contents of which are hereby incorporated by reference).

It will be understood by the skilled person that such further defined mutations can be achieved by using any conventional method as hereinbefore described but that in particular embodiments of the inventions the additional genetic mutation is provided by complete or partial deletion mutation or by insertional mutation.

Since the strains of the present invention have been found to be protective against infection by Francisella species in the mouse model of infection, the strains also provide useful vaccines against the diseases caused by Francisella infections and, in particular, tularemia. It is therefore preferred that the strains are formulated into pharmaceutical compositions, in which they are combined with a pharmaceutically acceptable carrier. Such pharmaceutical compositions form a second aspect of the invention. In this second aspect there is provided a pharmaceutical composition comprising a live strain of a Francisella species in which gene FTT1564, or homologue thereof, has been inactivated, and which is able to produce a protective immune response in an animal, in combination with a pharmaceutically acceptable carrier.

Suitable carriers may be solid or liquid carriers as is understood in the art. They may suitably be formulated for administration to mucosal surfaces (for example for oral use, of for administration by inhalation or insufflation) or for parenteral administration.

In particular they are formulated as sterile aqueous or oily solutions for intravenous, subcutaneous, intramuscular or intramuscular dosing.

Alternatively they are formulated for administration to mucosal surfaces and in particular for intranasal application.

Compositions are suitably prepared in unit dosage forms, as conventional in the art. They are administered at dosages which are determined using clinical practice, and depend upon factors such as the nature of the patient, the severity of the condition, and the precise vaccine strain being employed. Typically dosage units will comprise 10⁵-10⁸ cfu. Dosages may be boosted as appropriate or necessary.

Compositions may also contain further immunogenic reagents which are effective against F. tularensis infection or other diseases. They may further contain other agents such as adjuvants and the like, which enhance the host's immune response to the vaccine.

In particular embodiments of the second aspect of the invention, the live strain of the pharmaceutical composition comprises at least one additional genetic mutation, wherein said additional mutation inactivates any of the genes selected from the group of consisting of FTT0808, FTT0918, FTT0919 and homologues of these genes. In other embodiments the additional genetic mutation involves a gene which encodes for cell surface components or enzyme pathways utilised within the bacterial cell. Such further mutations include, but are not limited to, mutations which inactivate genes which encode the capsule component of the cell, such as capB and/or capC or pilin genes, for example mutations to pilA and/or pilE and/or pilC genes or other mutations such as those described in co-pending British patent application number GB0511722.1 (the contents of which are hereby incorporated by reference), mutations which inactivate genes which encode enzymes in the purine pathway, for example mutations to purA and/or purF genes and other purine pathway genes such as those described in co-pending International Application number PCT/GB2004/001264 (the contents of which are hereby incorporated by reference).

In a further aspect the present invention relates to the use of a strain of Francisella species in which gene FTT11564, or a homologue thereof, has been inactivated, and which is able to produce a protective immune response in an animal, in the preparation of a live prophylactic or therapeutic vaccine against infection by Francisella species.

In particular, such strains find use in the preparation, or manufacture, of a vaccine for the treatment of tularemia.

In yet a further aspect, the invention provides a method of preventing or treating infection caused by Francisella species, which method comprises administering to an animal, including a human being, an effective amount of a strain or of a pharmaceutical composition, each as hereinbefore described.

In particular, the method is useful in the treatment of infection caused by Francisella tularensis subspecies tularensis.

Novel strains which are suitable for vaccine use form a further aspect of the invention. In particular, the invention provides a strain of Francisella tularensis in which gene FTT1564, or a homologue of this gene, has been inactivated. In particular the strains which are suitable for use as vaccines are as hereinbefore described. In a preferred embodiment the FTT1564 gene is inactivated in Francisella tularensis subspecies tularensis strain SchuS4 to produce a live vaccine strain. As described above, the gene may be conveniently inactivated by complete or partial deletion mutation.

The invention will now be described by way of non-limiting example, with reference to the accompanying diagrammatic drawings in which:

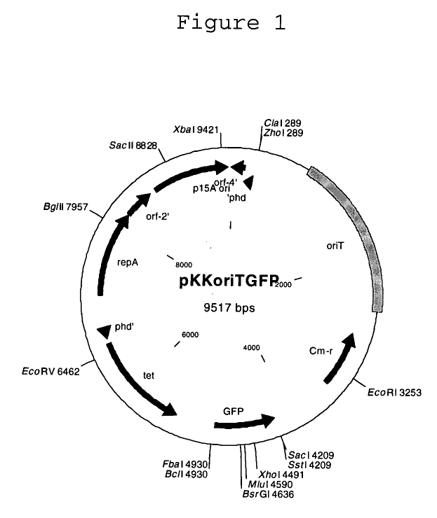

FIG. 1 shows the plasmid, designated pKKoriTGFP, generated to identify promoters induced during growth in macrophages.

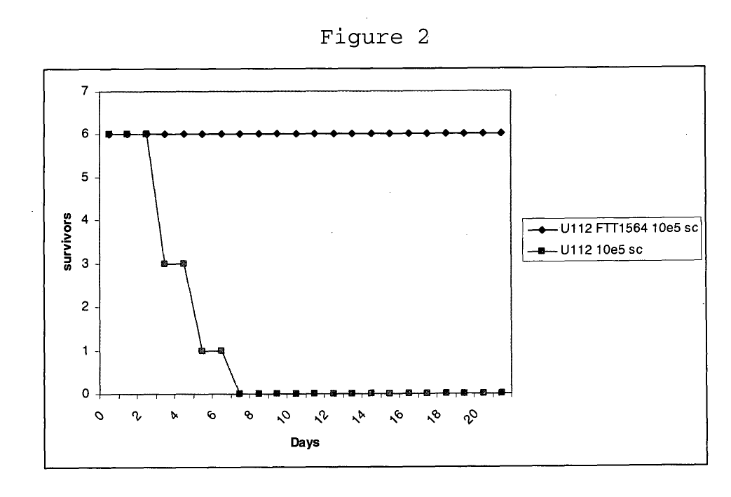

FIG. 2 shows the attenuation of F. novicida U112ΔFTT1564 i.e. a mutant in F. novicida in which the homologous gene to FTT1564 has been inactivated (i.e. FTN1472) (as compared with wild type F. novicida U112) in the mouse model of infection.

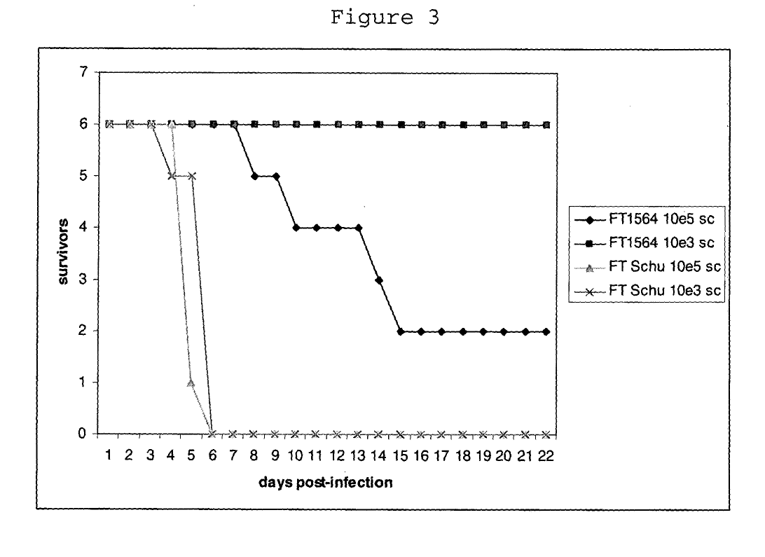

FIG. 3 shows the attenuation of F. tularensis SCHU S4ΔFTT1564 (as compared with fully virulent F. tularensis SCHU S4) in the mouse model of infection.

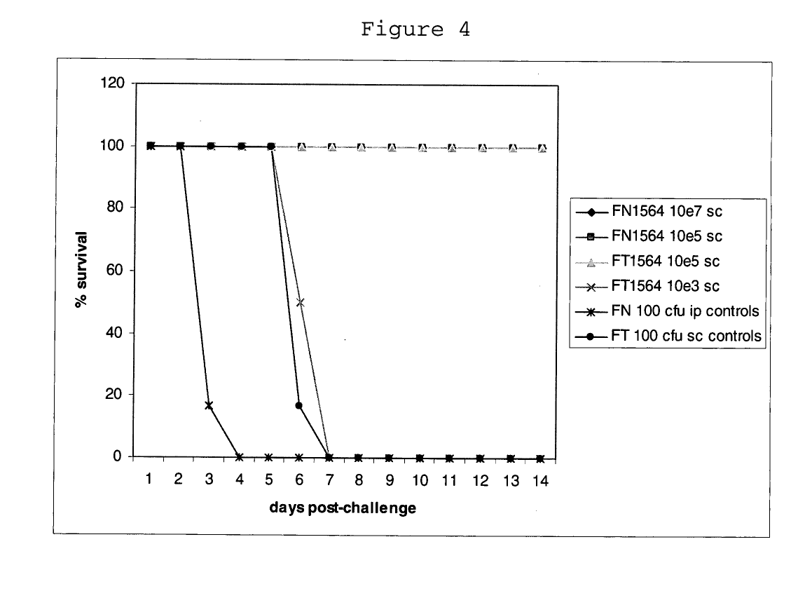

FIG. 4 shows survival data from mice, immunized with either F. novicida U112ΔFTT1564 (i.e. FTN1472) or F. tularensis SCHU S4ΔFTT1564 and subsequently challenged with virulent strains of F. novicida or F. tularensis.

### IDENTIFICATION OF PROMOTERS INDUCED DURING GROWTH IN MACROPHAGES

Promoter trapping using GFP (green fluorescent protein) expression was undertaken in order to identify Francisella genes which are induced intracellularly. The approach undertaken may be summarised in the following steps:
(i) Create promoter trap vector for Francisella
(ii) Create library of fragment inserts in F. novicida
(iii) Identify promoters induced in macrophages
(iv) Create mutants and characterise

A promoter trap vector was created as follows. The plasmid pKK202 is stably maintained in F. novicida. To facilitate conjugation, the oriT locus was subcloned into pKK202 from pPV2 to create pKK202oriT. The gene encoding GFP was amplified from pGFPuv (Clontech) using the polymerase chain reaction. The oligonucleotide primers were designed to include ApaLI restriction sites, allowing the amplified gene to be cloned into pKKoriT which had been linearized with that restriction enzyme. This plasmid was designated pKKoriTGFP, as shown in FIG. 1.

### Production of Libraries

Genomic DNA was isolated from F. novicida strain U112 and partially digested with Sau3AI (Roche). The DNA fragments were separated on a 0.7% agarose gel and the region covering <2 kb selected. The DNA was purified from the gel and ligated into pKKoriTGFP at the Fbal site. This process was repeated several times to generate different libraries. The plasmids were introduced into E. coli S17λ-pir for conjugation into F. novicida U112. Clones were selected on Thayer-Martin agar supplemented with chloramphenicol (to identify clones containing the plasmid) and polymixin (to inhibit growth of the E. coli donor). Clones were picked onto BCGA medium supplemented with chloramphenicol. Colonies were examined under UV and fluorescent colonies were removed from the library.

### Identification of Promoters Induced in Macrophages

The library was grown on BCGA agar supplemented with chloramphenicol, overnight at 37° C. Growth was removed from the agar using a sterile loop and the bacteria were resuspended in L15 tissue culture medium. J774.1 macrophages were infected at an MOI of 10, with an incubation of 30 min at 37° C. After incubation, extracellular bacteria were killed by the addition of L15 containing 10 μg/ml gentamycin for 30 min. This medium was replaced with L15 supplemented with 2 μg/ml gentamycin, and the macrophage assay was incubated overnight. Macrophages were examined for fluorescence using a confocal microscope. Fluorescent pools were broken down to single clones to identify the fluorescent clones.

Plasmids were isolated from positive clones and the inserts identified by genome sequencing.

TABLE 1
Genes of F. novicida induced in macrophages identified

| Gene designation | Proposed function |
|---|---|
| FTT0221 (acpA) | Acid phospatase |
| FTT0454 (yfdH) | Glycosyl transferase |
| FTT0689 | Unknown |
| FTT1269c (dnaK) | Chaperone - heat shock protein |
| FTT1443c | ATPase |
| FTT1564 | Unknown |
| FTT1736c (kdpD) | Kinase - two component system |

### Preparation of Mutants and Virulence Studies

To determine whether the genes identified in Table 1 were involved in virulence, a deletion mutant of one of the genes of unknown function, FTT1564, was constructed in F. novicida using routine methodology. The mutant was assessed for attenuation in the mouse model of infection. The LD₅₀ for strain U112 is approximately 10³ cfu by the intradermal route. 6 Balb/c mice were inoculated by the subcutaneous route with either strain U112 or U112ΔFTT1564. As can be seen in FIG. 2, F. novicida U112ΔFTT1564 is attenuated in the mouse model of infection with all six mice surviving until the end of the experiment (22 days) compared with no survivors from the group of mice inoculated with the virulent U112 strain (no survivors by day 7).

This approach has allowed the identification of genes not known previously to be induced intracellularly in Francisella. Inactivation of one of these genes resulted in attenuation in the mouse model.

A mutant was also produced in F. tularensis subspecies tularensis strain SCHU S4. The LD50 for strain SCHU S4 by the sc route is approximately 1 cfu. Mice were inoculated sc with either SCHU S4 or SCHU S4ΔFTT1564. As shown in FIG. 3, F. tularensis SCHU S4ΔFTT1564 is also attenuated in the mouse model, with 100% survivors at the end of the experiment (at an inoculation dose of 1000×LD₅₀).

### Protection Studies with Mutants Defective in FTT1156, or Homologue Thereof

Survivors from the virulence/attenuation studies described above were challenged with either 100 MLD F. novicida by the ip route, or 100 cfu SCHU S4 by the sc route. As is clearly shown in FIG. 4, the FTT1564 mutant in F. tularensis and the corresponding mutant in F. novicida provide complete protection against virulent strains of Francisella species. These attenuated mutants are of use in providing protection against tularemia.

In summary, Fu1564 was identified by trapping the promoter in a screen in macrophages. The function of the protein encoded by FTT1564 is not known. Inactivation of FTT11564 resulted in attenuation of two strains of Francisella. The F. novicida U112ΔFTT1564 mutant was able to induce solid protection against homologous challenge. The F. tularensis SCHU S4AFTT1564 induced 100% protection in survivors (from the attenuation study) at 10⁵ cfu delivered subcutaneously (sc).

1. A composition comprising a strain of Francisella species in which gene FTT1564, or a homologue thereof, is inactivated.

2. The composition of claim 1 wherein the gene is inactivated by complete or partial deletion mutation or by insertional mutation.

3. The composition of claim 1 wherein the strain is a strain of Francisella tularensis.

4. The composition of claim 3 wherein the strain is a strain of Francisella tularensis subspecies novicida.

5. The composition of claim 3 wherein the strain is a strain of Francisella tularensis subspecies tularensis.

6. The composition of claim 5 wherein the strain is Francisella tularensis subspecies tularensis strain SchuS4.

7. The composition of claim 1 wherein the strain further comprises at least one additional genetic mutation, wherein the additional mutation inactivates a gene selected from the group consisting of FTT0808, FTT0918, FTT0919 and homologues of these genes.

8. The composition of claim 1 wherein the strain further comprises at least one additional genetic mutation wherein the additional genetic mutation inactivates a gene that encodes for a capsule component of a cell, encodes for a pilin subunit or encodes for an enzyme in the purine pathway.

9. The composition of claim 8 wherein the additional genetic mutation inactivates a gene selected from the group consisting of capB, capC, pilA, pilE, pilC, purA and purF.

10. The composition of claim 7 wherein the additional genetic mutation is provided by complete or partial deletion mutation or by insertional mutation.

11. The composition of claim 1 further comprising a pharmaceutically acceptable carrier.

12-27. (canceled)

28. A method of preventing or treating infection caused by Francisella species comprising administering to an animal an effective amount of the pharmaceutical composition of claim 1.

29. (canceled)

30. The method of claim 28 wherein the strain is Francisella tularensis subspecies tularensis SchuS4.

31. The method of claim 28 wherein the gene is inactivated by complete or partial deletion mutation.
